(19) United States
(12) Patent Application Publication
(10) Pub. No.: US 2020/0113615 A1
(43) Pub. Date: Apr. 16, 2020

(54) DEVICE FOR HUMAN MEDICAL AND VETERINARY TREATMENT AND METHOD FOR GENERATING REACTIVE GAS THAT CAN BE USED IN PLASMA THERAPY

(71) Applicant: Lohmann & Rauscher GmbH, Wien (AT)

(72) Inventors: Patricia BUCHEGGER, Katzelsdorf (AT); Wolfgang HARREITHER, Traiskirchen (AT)

(21) Appl. No.: 16/603,994

(22) PCT Filed: Apr. 10, 2018
(86) PCT No.: PCT/EP2018/059152

§ 371 Date: Oct. 9, 2019

(30) Foreign Application Priority Data
Apr. 11, 2017 (DE) 10 2017 003 526.1

(51) Int. Cl. A61B 18/04; H05H 1/24; H05H 1/48; A61L 2/00; A61L 2/14

(52) U.S. Cl. CPC A61B 18/042; H05H 1/2406; A61B 2018/00452; A61L 2/0011; A61L 2/14; H05H 1/48

(57) ABSTRACT

A device for human medical or veterinary treatment comprising a gas discharge generator designed to generate reactive gases that can be used in plasma therapy in a gas discharge zone, a flow generator designed to generate a flow of gas from the gas discharge zone through a reaction chamber in the direction of an outlet opening of the reaction chamber, and an application device coupled to the outlet opening for discharging the reactive gases from the reaction chamber to the application location, the flow generator and/or the reaction chamber being designed to generate a turbulent flow in the reaction chamber and/or the gas discharge zone.

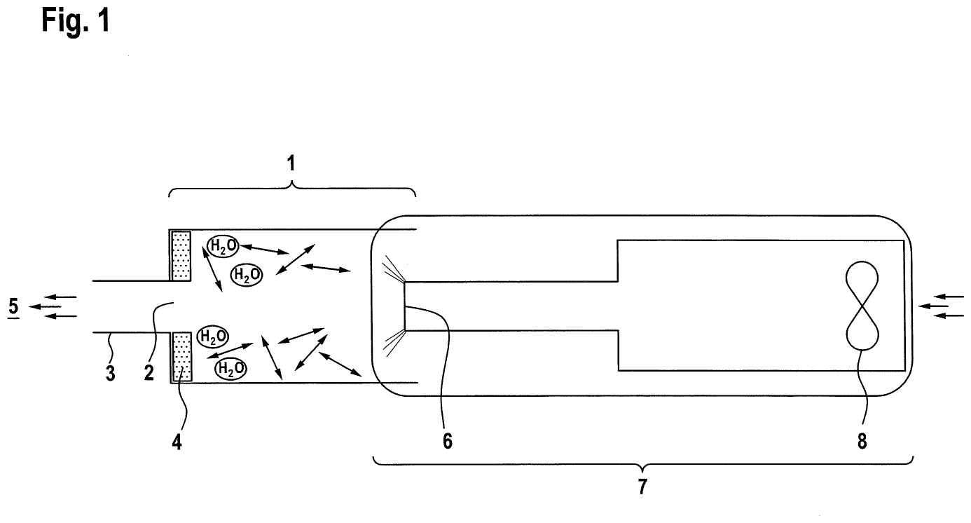

### CROSS REFERENCE TO RELATED APPLICATION

The present application is a 35 U.S.C. § 371 national phase entry application of, and claims priority to, International Patent Application No. PCT/EP2018/059152, filed Apr. 10, 2018, which claims priority to German Patent Application No. DE 102017003526.1, filed Apr. 11, 2017, the disclosures of which are hereby incorporated by reference in their entirety for all purposes.

### BACKGROUND

The invention relates to a device for human medical or veterinary treatment as specified in the pre-characterizing portion of claim 1.

In therapeutic treatment, in addition to conventional pharmaceutical products and physical treatment approaches, such as for example compression and/or negative pressure therapy, more recently treatment approaches have also been used in which reactive gases are used. Corresponding treatment approaches have become known in the field of dermatology for the treatment of dermatoses and wounds of any type, of oncological diseases and in dentistry. Over the course of plasma treatment reactive gas species are formed and are enriched specifically over the treatment area. This constitutes one possible physical treatment approach.

Reactive gases contain highly reactive components. These display their oxidative potential on the surface where they oxidise proteins, lipids and nucleic acids non-selectively. Higher eukaryotic cells have highly developed defence mechanisms and can deal with the oxidative stress that is caused substantially better than bacteria, fungi or viruses. These defence mechanisms include, among other things, antioxidants, enzymes that destroy ROS (Reactive Oxygen Species), such as catalases or dismutases, and DNA repair machinery. Therefore, reactive gases can be successfully used in particular in the treatment of wounds and dermatoses.

During plasma treatment the reactive gases are generally formed by transferring sufficient energy in a gas discharge. In this type of gas discharge a plasma composed of partially or fully charged particles is produced, from which reactive gas species develop. Plasma is often generated in electrostatic or electromagnetic fields, e.g. by alternating or direct current excitation or microwave excitation. For regenerative medical purposes cold atmospheric pressure plasma are generally used. If air is used as the reaction or process gas, reactive oxygen and nitrogen species, such as e.g. ozone (O₃) and hydrogen peroxide (H₂O₂) or nitric oxides with a strongly oxidizing effect are predominantly produced as products of the plasma. In addition, electrons and ions as well as photons emitted upon relaxation of the excited plasma components are produced in the plasma itself, which may themselves display an antiseptic effect that assists with wound healing.

Cold atmospheric pressure plasma can be formed by electric barrier discharge between insulation-coated electrodes by microwave excitation or a piezoelectric transformer as an active dielectric in a gas-filled space. In plasma therapy a basic distinction is made between the following treatment approaches:

1. Direct plasma therapy

In this therapy approach the plasma is generated in the treatment or wound area itself, generally by means of dielectric barrier discharge, the treatment area or the skin being able to be used as the electrode or counter-electrode in the plasma generation by means of gas discharge. Devices for direct plasma treatment are described, for example, in WO 2011/023478 and WO 2015/184395.

2. Indirect plasma therapy

In indirect plasma therapy the plasma is generated in a special plasma generator and conveyed by diffusion or optionally by means of compressed air, a pump or a ventilator to the treatment area. With the device for indirect plasma therapy described in DE 10 2012 003 563 A1 the reactive gases are generated in a plasma generator which is disposed in a housing. With the aid of a flow generator disposed within the housing the reactive gases are conveyed in the form of a free jet to the treatment area.

Further devices and methods for plasma therapy are disclosed in US 2013/199540 A1, US 2016/106993 A1, US 2003/139734 A1, KR 2016 0072759 A and US 2006/189976 A1.

With the conventional devices and methods for indirect plasma therapy it has been shown that in many cases satisfactory treatment results cannot be achieved.

### BRIEF DESCRIPTION OF THE DRAWINGS

The single FIGURE of the drawing shows a schematic illustration of a device according to the invention.

### DETAILED DESCRIPTION

In view of these problems in the prior art, the object underlying the invention is to provide devices and methods for indirect plasma therapy with which improved therapy results can be achieved.

With regard to the device, this object is achieved by a further development of the known devices as specified in the characterizing portion of claim 1.

With devices according to the invention the turbulent flow in the reaction chamber and/or the gas discharge zone leads to a buffer region being formed therein in which the individual molecules of the process gases are, as it were, intermediately stored, and over the duration of the intermediate storage are influenced by the electromagnetic fields of the gas discharge. The yield of reactive gases is thus increased by influencing the gas discharge. As a result, this leads to improved therapy success.

It has proven to be particularly advantageous if the outlet opening of the reaction chamber has a smaller opening area in a plane running perpendicular to the main flow direction of the gas flow generated by the flow generator than an inlet opening of the reaction chamber and/or the discharge zone. This gas flow aligned to the outlet opening then gives rise to dynamic pressure before the outlet opening. This dynamic pressure in turn leads to a turbulent flow guiding the process gases at least partially back into the discharge zone and creates the aforementioned buffer for the process gases by the process gases furthermore being subjected to the influence of the discharge.

In terms of the creation of defined flow ratios it has proven to be advantageous if the reaction chamber has a peripheral wall that is, at least in sections, in the form of a circular cylindrical jacket and at one end of which an inlet opening for the process gases can be provided, and at the other end of which the outlet opening is located. The cylinder axis of the peripheral wall section made in the form of a circular cylindrical jacket preferably runs approximately parallel to the main flow direction of the gas flow generated by the flow generator.

In terms of providing a simple and reliable application device it has proven to be advantageous if the outlet opening of the reaction chamber passes through a connecting piece made in the form of a tube coupling and preferably running approximately parallel to the peripheral wall in the form of a circular cylindrical jacket, to which connecting piece an application tube of the application device can be fitted.

In terms of avoiding an excessively great flow resistance while at the same time ensuring a sufficiently turbulent flow by means of which the buffering of the process gases can be ensured to a sufficient degree, it has proven to be advantageous if the ratio of the opening area of the inlet opening or the area of the gas discharge zone in a direction running perpendicular to the main flow direction to the opening area of the outlet opening is greater than 5, preferably greater than 10, in particular assuming a value of 15 or more, but is less than 90, in particular less than 50, preferably 40 or less.

It has been shown empirically that the diameter of the reaction chamber is advantageously smaller than 20 mm, preferably smaller than 15 mm, but greater than 5 mm, preferably greater than 7.5 mm, in particular approximately 10 mm. If the diameter of the reaction chamber is too large, the influence of the process gases in the buffer zone formed by the reaction chamber becomes too small due to the gas discharge, while at the same time an undesired abreaction of the reactive gases may already take place in the reaction chamber. On the other hand, a diameter of the reaction chamber that is too small leads to an undesired pressure increase in the reaction chamber, and this may lead to excessive collisional quenching of the reactive gases.

For the reasons given above, it is also advantageous if the reaction chamber has a length of 15 mm or more, in particular 20 mm or more in the main flow direction of the gas flow generated by the flow generator, the length of the reaction chamber, however, preferably being less than 80 mm, in particular less than 60 mm. A reaction chamber length of approximately 40 mm has proven to be particularly advantageous.

In terms of the effective production of reactive gases it has furthermore proven to be advantageous if the ratio of the axial length of the reaction chamber to the maximum diameter of the reaction chamber is greater than 2, in particular greater than 3, but smaller than 10, in particular smaller than 5.

Within the framework of the invention the discharge zone may also be located at least partially within the reaction chamber. For this purpose a discharge electrode, optionally encased with a dielectric or a piezotransformer may be located in the reaction chamber as a dielectric electrode.

The reaction chamber itself is preferably made of a chemically inert material, such as for example of electrically insulating plastic. In devices according to the invention the discharge direction and/or the discharge type may be specifically steered by the integration of one or more dielectric and/or metal parts. For example, it is conceivable to locate at least one metal part in the region of an inner boundary surface of the reaction chamber. The use of a dielectric barrier in the region of the discharge electrode leads to dielectric barrier discharge, whereas e.g. a metal ring after the discharge zone may lead to an arc discharge in this direction. In terms of influencing the composition of the reactive gases it has proven to be particularly advantageous if a humidifying device is provided for humidifying gases in the discharge zone and/or in the flow direction before the discharge zone and/or in the flow direction after the discharge zone.

As already explained above in connection with the use of an outlet opening that passes through a connecting piece, within the framework of the invention it has proven to be particularly advantageous if the application device has a tube, in particular a gas-tight plastic tube. Due to the flexibility of this type of tube the reactive gases generated in the discharge zone and/or the reaction chamber can be brought to almost any application locations. In order to avoid undesired collisional quenching of the reactive gases during transportation by the application device it has proven to be advantageous if the ratio of the length to the internal diameter of the tube is more than 7, preferably more than 10 and/or less than 30, in particular less than 25. The ratio of the length of the tube to the diameter is chosen to correspond to the diameter of the outlet opening and the dynamic pressure that is set so that a laminar flow forms in the tube in which there is only a small collision probability for the reactive gases, as a result of which the collisional quenching can be largely suppressed. It has proven to be particularly advantageous if the tube has an internal diameter of the range of between 2 and 8 mm, in particular of approximately 4 mm.

As already explained above in connection with devices according to the invention, a method according to the invention for generating reactive gases that can be used in plasma therapy, in which reactive gases are generated in a discharge zone from process gases, in particular air, and are discharged in the direction of an outlet opening of a reaction chamber, is essentially characterised in that a turbulent gas flow is generated in the discharge zone and/or the reaction chamber.

Within the framework of generating a turbulent gas flow a dynamic pressure in the range between 5 and 50 Pa, preferably between 10 and 40 Pa, in particular 23 to 35 Pa is generated here.

A compromise between the enrichment of reactive gases on the one hand and the particle flow of reactive gases on the other hand is achieved if the gases are discharged from the reaction chamber at a flow speed of less than 20 l/min, preferably less than 10 l/min, but more than 2.5 l/min. With excessively slow discharge of the reactive gases from the reaction chamber the reactive gases are enriched particularly well, but the particle flow of reactive gases becomes so small overall that successful therapy can no longer be achieved to a sufficient degree. Furthermore, when a minimum flow speed of 2.5 l/minis set, one can regularly dispense with the use of additional cooling devices for the elements bringing about the plasma generation, such as for example electrodes or piezo transformers. On the other hand, excessively rapid discharge of reactive gases from the reaction chamber leads to an overly small concentration of reactive gases in the gas that is discharged, which in turn puts the therapy success at risk. For these reasons the aforementioned upper and lower limits for the flow speed are particularly advantageous.

Within the framework of methods according to the invention the composition of the reactive gases can be influenced if the process gases are humidified before, during and/or after the discharge zone. Air can particularly preferably be used as the process gas. The humidification of the process gases advantageously takes place such that the relative humidity of the process gases is lowered to less than 50%, in particular less than 15%. With strong humidification of the process gases, hydrogen peroxide is predominantly formed as the reactive gas, but with a concentration that is too high, this abreacts with water and oxygen. For this reason the relative humidity of the process gases is limited to less than 50%. Within the framework of the invention it has proven to be advantageous if the ambient air has been dried to a value of 10% relative humidity before being introduced into the discharge zone.

The single FIGURE of the drawing shows a schematic illustration of a device according to the invention. The device comprises a ventilator 8, with the aid of which air is conveyed into the region of an electrode 6. Gas discharge with plasma generation takes place in the region of the electrode 6. Connected downstream of the electrode 6 is a reaction chamber 1 in which the process gases are buffered and from which the process gases are discharged through an outlet opening 2. The reaction chamber is delimited by a wall in the form of a circular cylindrical jacket to the end of which facing away from the electrode 6 a front wall is attached in which a connecting piece 3 for a tube that can be used as an application device is attached. The internal diameter of the connecting piece 3 is smaller than the internal diameter of the wall of the reaction chamber 1 that is in the form of a circular cylindrical jacket. In this way a dynamic pressure can form on the front surface of the reaction chamber 1 facing away from the electrode 6, which pressure in turn causes a turbulent flow by means of which the process gases pass a number of times into the discharge region, i.e. into the region of the electrode 6.

In the embodiment of the invention illustrated by the drawing, an air humidification device made in the form of a pad is provided in the region of the front surface of the reaction chamber 1 facing away from the electrode 6, by means of which the composition of the reactive gases can be influenced. In the embodiment of the invention illustrated by the drawing a gas discharge zone forms in the region of the electrode 6, which zone reaches into the reaction chamber 1.

The invention is not restricted to the embodiment illustrated by the drawing. In a further development of this embodiment the use of piezoelectric transformers is also conceivable. With these transformers the greatest potential is at the corners on the front end. Therefore, discharge and plasma formation normally take place at these corners. The integration of metal parts into the reaction chamber before the gas discharge zone may additionally bring about a discharge along the edges. These additional parasitic discharges may increase the efficiency of the ionisation of the ambient air because more plasma can be produced in a shorter time.

### LIST OF REFERENCE NUMBERS

1 ionisation chamber

2 outlet constriction

3 tube coupling

4 pad for air humidity enrichment

5 multiply ionised reactive gas

6 plasma generation

7 plasma source

8 ventilator

9 input gas

1. A device for human medical or veterinary treatment comprising a gas discharge generator designed to generate reactive gases that can be used in plasma therapy in a gas discharge zone,
a flow generator designed to generate a flow of gas from the gas discharge zone through a reaction chamber in the direction of an outlet opening of the reaction chamber, and
an application device coupled to the outlet opening for discharging the reactive gases from the reaction chamber to the application location,
wherein the ratio of the opening area of the inlet opening or of the gas discharge zone to the opening area of the outlet opening is greater than 5, preferably greater than 10, in particular 15 or greater and the ratio of the opening area of the inlet opening or of the gas discharge zone to the opening area of the outlet opening is less than 90, in particular less than 50, preferably 40 or less, the flow generator and/or the reaction chamber is/are designed to generate a turbulent flow in the reaction chamber and/or the gas discharge zone.

2. (canceled)

3. The device according to claim 1, characterised in that the reaction chamber has a peripheral wall that is, at least in sections, in the form of an approximately circular cylindrical jacket, and at one end of which can be an inlet opening of the reaction chamber and at the other end of which the outlet opening is located.

4. The device according to claim 1, characterised in that the outlet opening passes through a connecting piece made in the form of a tube coupling and preferably running approximately parallel to the peripheral wall in the form of a circular cylindrical jacket.

5. (canceled)

6. (canceled)

7. The device according to claim 1, characterised in that the diameter of the reaction chamber is smaller than 20 mm, preferably smaller than 15 mm and/or greater than 5 mm, preferably greater than 7.5 mm, in particular approximately 10 mm.

8. The device according to claim 1, characterised in that the reaction chamber has a length of 15 mm or more, in particular 20 mm or more in the main flow direction of the gas flow generated by the flow generator, but less than 80 mm, in particular less than 60 mm, the length of the reaction chamber preferably being approximately 40 mm.

9. The device according to claim 1, characterised in that the ratio of the axial length of the reaction chamber to the maximum diameter is greater than 2, in particular greater than 3, but is smaller than 10, in particular is smaller than 5.

10. The device according to claim 1, characterised in that the discharge zone is located at least partially within the reaction chamber.

11. The device according to claim 1, characterised in that the reaction chamber is made, at least in sections, of insulating plastic.

12. The device according to claim 1, characterised that at least one metal part is located in the region of an inner boundary surface of the reaction chamber.

13. The device according to claim 1, characterised in that the gas discharge generator can be operated to generate a dielectric barrier discharge.

14. The device according to claim 1, characterised by a humidifying device for humidifying process gases in the discharge zone and/or in the flow direction before the discharge zone and/or in the flow direction after the discharge zone.

15. The device according to claim 1, characterised in that the application device has a tube, in particular a gas-tight plastic tube.

16. The device according to claim 15, characterised in that the ratio of the length to the internal diameter of the tube is more than 7, preferably more than 10 and/or less than 30, in particular less than 25.

17. The device according to claim 15, characterised in that the tube has an internal diameter in the range between 2 and 8 mm, in particular of approximately 4 mm.

18. (canceled)

19. (canceled)

20. (canceled)

21. (canceled)

22. (canceled)
